(19) United States
(12) Patent Application Publication
(10) Pub. No.: US 2025/0213297 A1
(43) Pub. Date: Jul. 3, 2025

(54) LONG-ELECTRODE CONCEPT FOR DISCRETE CUTTING WITH RF ENERGY IN ELECTROSURGICAL APPLICATIONS

(71) Applicant: Boston Scientific Medical Device Limited, Galway (IE)

(72) Inventors: Matthew DiCicco, Toronto (CA); Eduardo Moriyama, Richmond (CA); Christian Balkovec, Cambridge (CA); Gareth Davies, Toronto (CA)

(21) Appl. No.: 19/002,236

(22) Filed: Dec. 26, 2024

Related U.S. Application Data
(60) Provisional application No. 63/616,342, filed on Dec. 29, 2023.

(51) Int. Cl. A61B 18/14; A61B 18/00; A61M 25/06; A61M 25/09

(52) U.S. Cl. CPC A61B 18/1492; A61B 2018/00351; A61B 2018/00601; A61B 2018/144; A61M 25/065; A61M 2025/09183

(57) ABSTRACT

An electrosurgical device for discretely cutting target tissue that is adjacent non-target tissue includes an elongate shaft having a proximal portion including a proximal end and a distal portion including a distal end. At least one electrode is configured to cut the target tissue. The at least one electrode has a surface area and is located on the distal portion. The distal portion is electrically insulated from the at least one electrode and has a surface area significantly larger than the surface area of the at least one electrode. The distal portion is configured to prevent movement of the distal portion into a cut or puncture formed in the target tissue by application of energy to the at least one electrode.

### CROSS REFERENCE TO RELATED APPLICATIONS

This application claims priority to U.S. Provisional Patent Application No. 63/616,342 entitled “LONG-ELECTRODE CONCEPT FOR DISCRETE CUTTING WITH RF ENERGY IN ELECTROSURGICAL APPLICATIONS,” filed Dec. 29, 2023, which is hereby incorporated by reference in its entirety.

### TECHNICAL FIELD

The present disclosure relates to medical systems and methods for discretely cutting tissue in a patient. More specifically, the present disclosure relates to medical systems and methods for discretely cutting a target tissue that is apposed to a non-target tissue.

### BACKGROUND

The use of RF energy to cut or vaporize biological tissue is a widely used technique in surgical applications. A target tissue can be vaporized using a conductive material that delivers RF energy of a specific voltage, frequency, and current density to it. This can be used to ligate or cut tissue with a cautery pen in general electrosurgery or to access locations in the body or vasculature through non-invasive percutaneous surgery. During percutaneous transseptal access from the right to the left atrium in the heart, for example, it is common to apply RF energy to puncture the interatrial septum (IAS). In these cases, controlling the depth of tissue crossed is not necessary as beyond either side of the IAS is the open space of the right and left atrium.

In some applications, delivering RF energy to a precise tissue at a precise depth is desired. In the case of percutaneous access to the epicardial surface of the heart, for example, the pericardial sac that surrounds the outer surface of the heart must be punctured without advancing into the myocardium. Here the target tissue is apposed to non-target tissue where vaporization is not desired. In these cases, an electrosurgical tool that delivers RF energy precisely to ONLY the target tissue would greatly benefit both the user and patient, by reducing the risk of cutting, puncturing, vaporizing, or otherwise harming non-target tissue.

In order to perform cutting in electrosurgery, generally the electrode is designed to have a small surface area, on the order of 1-3 mm², surrounded by non-conductive material to provide sufficient current density at a desired voltage and frequency. In general surgery, the electrodes of electrocautery pens are designed to advance through or ligate target tissue indiscriminately, as the physician can visually see target and non-target tissue and can finely control the position and force applied to the tissue. For puncturing of the IAS percutaneously, the physician has much less direct control on the position and force of the RF probe but discrete cutting is not as important as there are typically no non-target tissues in apposition to the IAS.

Development of RF probes that promote discrete cutting of tissue in situations where physicians either have less control over the position and force of the electrode or cannot visually confirm the characteristics (e.g. thickness, pliability) may be advantageous. An electrode designed to be unable to directly advance through target tissue may reduce complications of indiscriminately cutting non-target tissue in these applications.

### SUMMARY

Example 1 is an electrosurgical device for discretely cutting target tissue that is adjacent non-target tissue. The electrosurgical device includes an elongate shaft having a proximal portion including a proximal end and a distal portion including a distal end. At least one electrode is configured to cut the target tissue. The at least one electrode has a surface area and is located on the distal portion. The distal portion is electrically insulated from the at least one electrode and has a surface area significantly larger than the surface area of the at least one electrode. The distal portion is configured to prevent movement of the distal portion into a cut or puncture formed in the target tissue by application of energy to the at least one electrode.

Example 2 is the electrosurgical device of Example 1, wherein the elongate shaft includes a lumen extending from the proximal end to the distal end.

Example 3 is the electrosurgical device of any of Examples 1 or 2, wherein the at least one electrode is formed of a wire having one or more rectangular, circular, oval, square, or polygonal cross-section.

Example 4 is the electrosurgical device of any of Examples 1-3, wherein the at least one electrode is formed of an electrically conductive metal.

Example 5 is the electrosurgical device of any of Examples 1-4, further comprising at least one lead configured to electrically connect the at least one electrode to a control system.

Example 6 is the electrosurgical device of Example 5, wherein the at least one lead extends proximally from the at least one electrode through a lead lumen to the proximal end of the elongate shaft.

Example 7 is the electrosurgical device of Example 5, wherein the at least one lead forms the at least one electrode.

Example 8 is the electrosurgical device of any of Examples 1-7, further comprising a connector located at the proximal end configured for electrically connecting the electrosurgical device to a control system.

Example 9 is the electrosurgical device of any of Examples 1-8, wherein the at least one electrode extends longitudinally along the outer surface of the distal portion.

Example 10 is the electrosurgical device of Example 9, wherein the outer surface tapers towards the distal end.

Example 11 is the electrosurgical device of any of Examples 1-10, wherein the at least one electrode is located in a recess or groove along the distal portion.

Example 12 is the electrosurgical device of any of Examples 1-11, wherein the at least one electrode extends along a longitudinal axis of the elongate shaft.

Example 13 is the electrosurgical device of any of Examples 1-12, wherein the at least one electrode has a surface area less than 1.0 mm².

Example 14 is the electrosurgical device of any of Examples 1-13, wherein the at least one electrode is located on a distal face of the elongate shaft.

Example 15 is the electrosurgical device of any of Examples 1-14, wherein the at least one electrode comprises a plurality of electrodes.

Example 16 is an electrosurgical device for discretely cutting pericardium that is adjacent myocardium. The electrosurgical device includes an elongate shaft having a proximal portion including a proximal end and a distal portion including a distal end. At least one electrode is configured to cut the pericardium. The at least one electrode includes a surface area and is located on the distal portion. The distal portion is electrically insulated from the at least one electrode and has a surface area significantly larger than the surface area of the at least one electrode. The distal portion is configured to prevent movement of the distal portion into a cut or puncture formed in the pericardium by application of energy to the at least one electrode.

Example 17 is the electrosurgical device of Example 16, wherein the elongate shaft includes a lumen extending from the proximal end to the distal end.

Example 18 is the electrosurgical device of Example 16, wherein the at least one electrode is formed of a wire having one or more rectangular, circular, oval, square, or polygonal cross-section.

Example 19 is the electrosurgical device of Example 16, wherein the at least one electrode is formed of an electrically conductive metal.

Example 20 is the electrosurgical device of Example 16, further comprising at least one lead configured to electrically connect the at least one electrode to a control system.

Example 21 is the electrosurgical device of Example 20, wherein the at least one lead extends proximally from the at least one electrode through a lead lumen to the proximal end of the elongate shaft.

Example 22 is the electrosurgical device of Example 20, wherein the at least one lead forms the at least one electrode.

Example 23 is the electrosurgical device of Example 16, further comprising a connector located at the proximal end configured for electrically connecting the electrosurgical device to a control system.

Example 24 is the electrosurgical device of Example 16, wherein the at least one electrode extends longitudinally along the outer surface of the distal portion.

Example 25 is the electrosurgical device of Example 24, wherein the outer surface tapers towards the distal end.

Example 26 is the electrosurgical device of Example 16, wherein the at least one electrode is located in a recess or groove along the distal portion.

Example 27 is the electrosurgical device of Example 16, wherein the at least one electrode has a surface area less than 1.0 mm².

Example 28 is the electrosurgical device of Example 16, wherein the at least one electrode is located on a distal face of the elongate shaft.

Example 29 is the electrosurgical device of Example 16, wherein the at least one electrode comprises a plurality of electrodes.

Example 30 is an electrosurgical device for discretely cutting pericardium that is adjacent myocardium. The electrosurgical device includes an elongate shaft having a proximal portion including a proximal end and a tapered distal portion including a distal end. At least one electrode is configured to cut the pericardium. The at least one electrode includes a surface area and extends longitudinally along an outer surface of the distal portion. The distal portion is electrically insulated from the at least one electrode and has a surface area significantly larger than the surface area of the at least one electrode. The distal portion is configured to prevent movement of the distal portion into a cut or puncture formed in the pericardium by application of energy to the at least one electrode.

Example 31 is a method for discretely cutting target tissue using radiofrequency energy. The method includes providing an electrosurgical device. The electrosurgical device includes an elongate shaft having a proximal portion including a proximal end and a distal portion including a distal end. At least one electrode is configured to cut the target tissue. The at least one electrode has a surface area and extends longitudinally along an outer surface of the distal portion. The distal portion is electrically insulated from the at least one electrode and has a surface area significantly larger than the surface area of the at least one electrode. The method includes advancing the electrosurgical device to a target location within a patient. The method includes providing radiofrequency energy to the at least one electrode and forming a cut in the target tissue. The distal portion contacts tissue adjacent the cut in order to prevent movement of the distal portion into the cut.

Example 32 is the method of Example 31, wherein the distal portion tapers towards the distal end.

Example 33 is the method of Example 31, wherein the elongate shaft includes a lumen extending from the proximal end to the distal end.

Example 34 is the method of Example 31, wherein the target tissue is pericardium.

Example 35 is the method of Example 34, further comprising advancing a guidewire through the cut into a pericardial space.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a perspective view of an electrosurgical device for discretely cutting tissue, in accordance with an embodiment of the disclosure.

FIG. 2 is a perspective view of an electrosurgical device for discretely cutting tissue, in accordance with an embodiment of the disclosure.

FIGS. 3A-3E illustrate various cross-sections for wires or leads forming RF electrodes, in accordance with the disclosure.

FIGS. 4A-4C illustrate various cross-sectional views of an electrosurgical device showing various arrangements for an RF electrode, in accordance with the disclosure.

FIGS. 5A-5C illustrate various views of a distal end of an electrosurgical device showing various arrangements for an RF electrode, in accordance with the disclosure.

FIGS. 6A-6D illustrate a method of creating a pericardial puncture and accessing the pericardial space, in accordance with the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

Minimally invasive access to the pericardial space is required for diagnosis and treatment of a variety of arrhythmias and other conditions. Access to the space may be initiated using a large diameter (for example, about 17 Ga) Tuohy-style needle via the subxiphoid approach. A guidewire (for example, about 0.032 inches (about 0.81 mm) in outer diameter) is then advanced to the heart through the needle lumen. After gaining access to the pericardial space, the operator removes the Tuohy-needle then advances and secures a sheath (for example, 8.5 Fr) to facilitate use of treatment devices such as ablation and mapping catheters.

Mechanical puncture using large bore needles, as described above, is associated with a high clinical complication rate. Although the stiff needle provides some stability and some tactile feedback to the user, unwanted tissue damage is possible if the needle inadvertently punctures or unintentionally lacerates tissue.

As a consequence of the challenges and uncertainties of using mechanical puncture for accessing the pericardial space, physicians may resort to common endocardial ablation in situations where epicardial ablation is a preferred treatment, such as ventricular tachycardias. New devices or methods to improve the safety and predictability of gaining access to the pericardial space would be of benefit.

In the case of epicardial access, an electrosurgical probe can be advanced through the thoracic cavity to the pericardial sac that surrounds the heart. This is in apposition to the myocardium (i.e. external surface) of the heart. By increasing the surface area of non-conductive elements around an electrode, the amount of force applied by the cutting surface of the electrode is effectively reduced. This results in the cutting of only target tissue that is directly in-contact with the electrode while preventing the electrode from inadvertently advancing due to a “release” in force build-up. The large surface area of material surrounding the electrode prevents advancement of the electrode through tissue regardless of a pressure applied to the device by a user. This allows for vaporization of tissue immediately surrounding the electrode by preventing advancement of the electrode beyond the target tissue.

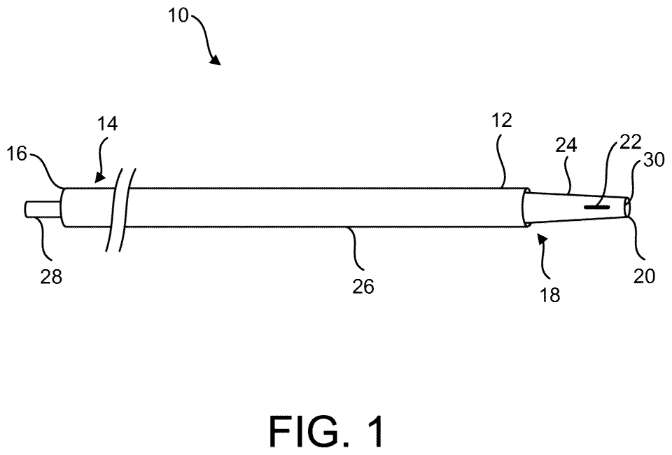

FIG. 1 is a perspective view of an electrosurgical device 10 for discretely cutting tissue, in accordance with an embodiment of the disclosure. In one aspect, the electrosurgical device 10 is configured to discretely cut target tissue that is adjacent non-target tissue. The target tissue can include any tissue located in a body that is desire to be cut, sliced, punctured, pierced, or otherwise modified. The electrosurgical device 10 includes an elongate shaft 12 having a proximal portion 14 including a proximal end 16 and a distal portion 18 including a distal end 20.

In one embodiment, the elongate shaft 12 is formed of an insulative material. Insulative materials may include a polymeric material or a ceramic material. Polymeric materials may include polyetheretherketone, epoxy, polyurethane, or parylene, for example. Ceramic materials may be deposited, fired, molded, and/or machined. In another embodiment, the elongate shaft 12 is formed of a conductive material such as include MP35N, Elgiloy, MP35N LT, platinum alloys, stainless steel alloys, palladium alloys, and titanium. In this configuration, an insulative sheath or coating 26 surrounds the conductive shaft 12.

The electrosurgical device 10 includes at least one RF electrode 22. The at least one RF electrode 22 is configured to cut a target tissue by the application of RF energy to the tissue. The at least one RF electrode 22 is located on the distal portion 18 of the elongate shaft 12, and is electrically insulated from the distal portion 18. In some embodiments, the distal portion 18 includes a tapered section 24 that decreases in diameter towards the distal end 20. This allows the electrosurgical device 10 to act as a dilator to stretch or dilate a puncture, slice, or cut formed by the at least one RF electrode 22. The at least one electrode 22 can take the form of a long, thin electrode that is located along a portion of the length the distal portion 18.

The at least one electrode 22 is configured to vaporize tissue upon application of RF energy to the electrode in order to form a puncture, slice, or cut within the tissue. The proximal portion 14 of the elongate shaft 12 includes a connector 28 configured to electrically connect the electrosurgical device 10 to a control system for controlling delivery of RF energy to the at least one electrode 22.

The at least one RF electrode 22 extends longitudinally along the outer surface of the distal portion 18 and has a surface area that is significantly smaller than a surface area of the distal portion 18 of the elongate shaft 12 adjacent the at least one RF electrode 22. For example, the at least one RF electrode 22 has a surface area less than 1.0 mm². The significantly larger surface area of the distal portion 18 reduces movement of the distal portion 18 into a cut or puncture formed in the target tissue. This configuration prevents inadvertent crossing the at least one RF electrode 22 and the distal portion 18 into any tissue immediately apposed to target tissue.

A lumen 30 extends from the proximal end 16 of the elongate shaft 12 to the distal end 20 of the elongate shaft 12. In one aspect, the lumen 30 extends through the connector 28. The lumen 30 is configured for introduction of a fluid or a medical device into the electrosurgical device 10 for delivery out of the distal end 20. For example, a contrast medium may be delivered from the distal end 20 or a guidewire can be extended from the lumen 30.

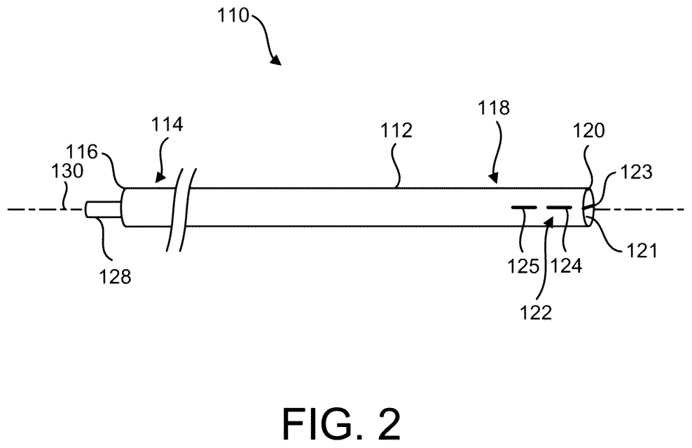

FIG. 2 is a perspective view of an electrosurgical device 110 for discretely cutting tissue, in accordance with an embodiment of the disclosure. The electrosurgical device 110 includes an elongate shaft 112 having a proximal portion 114 including a proximal end 116 and a distal portion 118 including a distal end 120. The distal portion 118 includes a substantially uniform diameter. The distal end 120 includes a distal face 121. As discussed above, the elongate shaft 112 can be formed of an insulative material or a conductive material that is covered in an insulative coating or sheath.

The electrosurgical device 110 includes at least one RF electrode 122 located on the distal portion 118 of the elongate shaft 112. The at least one RF electrode 122 is electrically insulated from the distal portion 118. The at least one RF electrode 122 includes a first electrode 123 located on the distal face 121, and a second electrode 124 and a third electrode 125 located on an outer surface of the distal portion 118.

Each of the first electrode 123, second electrode 124, and third electrode 125 has a surface area that is significantly smaller than a surface area of portion of the elongate shaft 112 adjacent thereof. The significantly larger surface area of the adjacent portion prevents movement of the distal portion 118 into a cut or puncture formed in the target tissue after forming a cut or puncture by application of energy to any of the first electrode 123, second electrode 124, and third electrode 125. In one aspect, RF energy can be delivered to each of the first electrode 123, second electrode 124, or third electrode 125 independently. As such, a user can select which electrode to actuate to perform various portions of a procedure. In another aspect, RF energy is delivered to each of the first electrode 123, second electrode 124, or third electrode 125 simultaneously.

The first electrode 123 is positioned across the distal face 121 intersecting with the center of the distal face 121. In some embodiments, the first electrode 123 resides in a plane common with the second electrode 124 and the third electrode 125. In some embodiments, the first electrode 123 resides in a plane orthogonal or offset from a plane in which the second electrode 124 and the third electrode 125 reside. In some embodiments, the first electrode is positioned across the distal face 121 offset from the center thereof.

The second electrode 124 and the third electrode 125 extend longitudinally along the outer surface of the distal portion 118 parallel to or adjacent a longitudinal axis 130 of the elongate shaft 112. In some embodiments, more than two electrodes are located along the distal portion 118. Additionally, in some embodiments, either the second electrode 124, the third electrode 125, or both the second electrode 124 and the third electrode 125 extend across the outer surface of the distal portion 118, offset from or orthogonal to the longitudinal axis 130 of the elongate shaft 112.

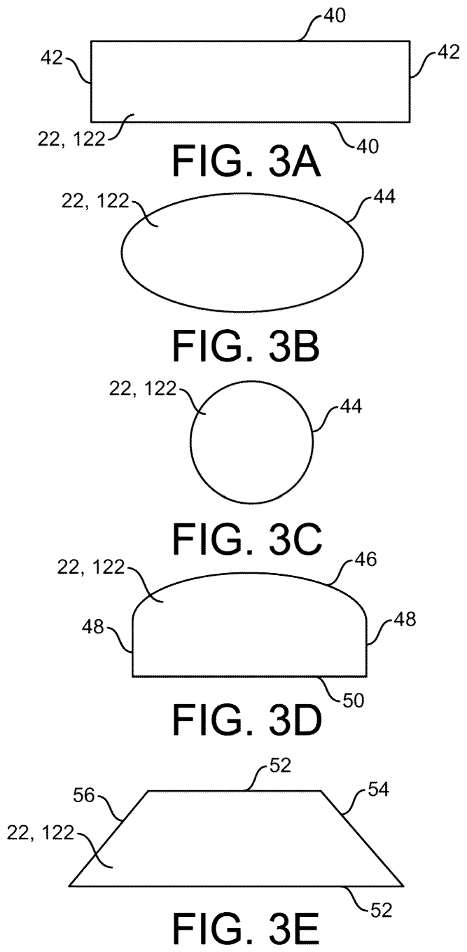

FIGS. 3A-3E illustrate various cross-sections for wires, filaments, or leads forming at least one RF electrode 22, 122, in accordance with the disclosure. The wires, filaments, or leads forming the at least one RF electrode 22, 122 can include a variety of cross-sections and can be formed of a conductive material. In some embodiments, the wires or leads can include copper, MP35N, Elgiloy, MP35N LT, platinum alloys, stainless steel alloys, palladium alloys, titanium, and combinations thereof. In some embodiments, the wires, filaments, or leads may include a conductive material that is radiopaque. The wires, filaments, or leads are configured to be located on a surface of the elongate shaft 12, 112 to form the at least one RF electrode 22, 122. In one aspect, the wires, filaments, or leads form the at least one RF electrode 22, 122 and extend from the at least one RF electrode 22, 122 to the connector 28, 128. In another aspect, a separate conductive wire or lead extends from the at least one RF electrode 22, 122 to the connector 28, 128.

FIG. 3A illustrates a wire, filament, or lead forming at least one RF electrode 22, 122 having a rectangular cross-section. The rectangular cross-section includes a first pair of surfaces 40 that are orthogonal to a second pair of surfaces 42. FIG. 3B illustrates a wire, filament, or lead forming at least one RF electrode 22, 122 having an oval cross-section. The oval cross-section includes a single surface 44. FIG. 3C illustrates a wire or lead forming at least one RF electrode 22, 122 having a circular cross-section. Like the oval cross-section, the circular cross-section includes a single surface 44. FIG. 3D illustrates a wire, filament, or lead forming at least one RF electrode 22, 122 having a dome shaped cross-section. The dome shaped cross-section includes a curved surface 46, and a first pair of parallel surfaces 48 that are orthogonal to a flat surface 50 opposite of the curved surface 46. FIG. 3E illustrates a wire, filament, or lead forming at least one RF electrode 22, 122 having a polygonal cross-section. The polygonal cross-section includes a pair of parallel surfaces 52 that are intersected by a first angled surface 54 and a second angled surface 56.

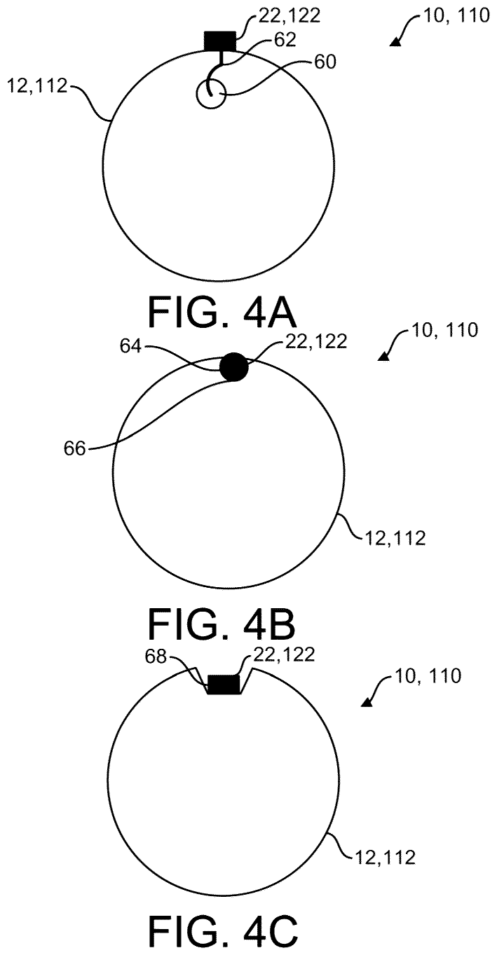

FIGS. 4A-4C illustrate various cross-sectional views of an electrosurgical device 10, 110 showing various arrangements for at least one RF electrode 22, 122 in accordance with the disclosure. In FIG. 4A, the at least one RF electrode 22, 122 is mounted to an outer surface of the elongate shaft 12, 112. As can be seen, the surface area of the at least one RF electrode 22, 122 is significantly smaller than the surrounding surface area of the elongate shaft 12, 112. This allows for the at least one RF electrode 22, 122 to create a puncture or cut in a target tissue without any portion of the elongate shaft 12, 112 pressing completely into the puncture or cut. This protects non-target tissue that is adjacent the target tissue. FIG. 4A illustrates an example of a lead lumen 60 configured to receive a lead 62 for electrically connecting the at least one RF electrode 22, 122 to a control system. The lead 62 extends proximally from the at least one RF electrode 22, 122 through the lead lumen 60 to the proximal end 16, 116 of the elongate shaft 12, 122 to connector 28, 128. In some aspects, the lead 62 is the same wire or filament forming the at least one RF electrode 22, 122. As such, the lead 62 includes a cross-section identical to the cross-section of the at least one RF electrode 22, 122. In some aspects, the lead 62 is a different wire or filament from a wire or filament forming the at least one RF electrode 22, 122.

In FIG. 4B, the at least one RF electrode 22, 122 is partially embedded into the elongate shaft 12, 112 of the electrosurgical device 10, 110. In this arrangement, the at least one RF electrode 22, 122 has a portion that extends partially above (at 64) and partially below (at 66) the outer surface of the elongate member 12. The at least one RF electrode 22, 122 can be positioned partially in a groove or channel located on the outer surface of the elongate shaft 12, 112.

In FIG. 4C, the at least one RF electrode 22, 122 is positioned entirely within a channel or groove 68 on the elongate member of the electrosurgical device 10, 110. In this arrangement, the at least one RF electrode 22, 122 remains entirely below the outer surface of the elongate member 12, 112. In this arrangement, tissue must partially enter the channel or groove 68 in order to be punctured or cut by the at least one RF electrode 22, 122.

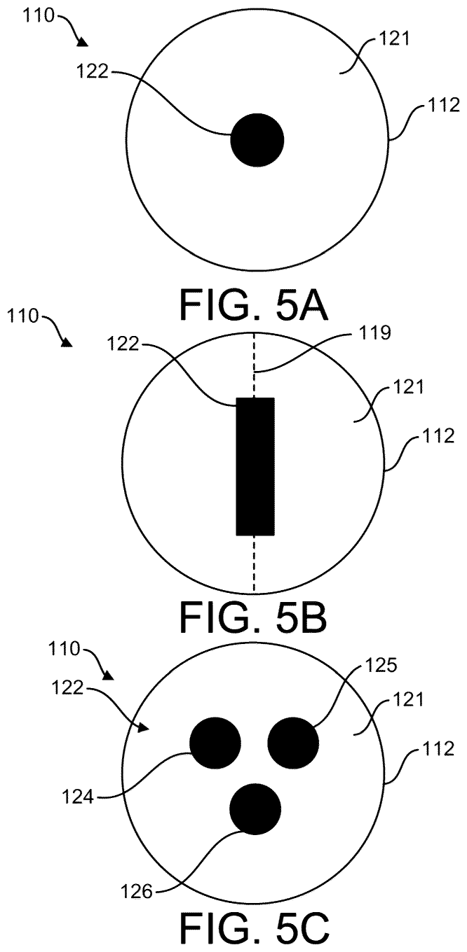

FIGS. 5A-5C illustrate various plan views of a distal face 121 of an electrosurgical device 110 showing various arrangements for at least one RF electrode 122, in accordance with the disclosure. In FIG. 5A, the at least one RF electrode 122 is positioned at the center of the distal face 121. The at least one RF electrode 122 is formed by a portion of an exposed wire, filament, or lead having a circular cross-section. The at least one RF electrode 122 has a surface area that is significantly smaller than the surface area of the distal face 121 surrounding the at least one RF electrode 122. The distal face 121 is insulated or non-electric such that the only portion of the electrosurgical device 110 that forms a cut or puncture in tissue is the at least one RF electrode 122. The larger surface area of the distal face 121 prevents entry of the electrosurgical device 110 into a cut or puncture formed by the at least one RF electrode 122.

In FIG. 5B, the at least one RF electrode 122 is positioned such that it transverses the distal face 121 along a central axis 119 of the distal face 121. The at least one RF electrode 122 has a surface area that is significantly smaller than the surface area of the distal face 121. The distal face 121 is insulated or non-electric such that the only portion of the electrosurgical device 110 that forms a cut or puncture in tissue is the at least one RF electrode 122. The larger surface area of the distal face 121 prevents entry of the electrosurgical device 110 into a cut or puncture formed by the at least one RF electrode 122.

In FIG. 5C, the at least one RF electrode 122 includes a plurality of RF electrodes 124, 125, 126 that are positioned uniformly around the center of the distal face 121. The plurality of RF electrodes 124, 125, 126 have a combined surface area that is significantly smaller than the surface area of the distal face 121. The distal face 121 is insulated or non-electric such that the only portion of the electrosurgical device 110 that forms a cut or puncture in tissue is the plurality of RF electrodes 124, 125, 126. The larger surface area of the distal face 121 prevents entry of the electrosurgical device 110 into a cut or puncture formed by the plurality of RF electrodes 124, 125, 126. While three RF electrodes 124, 125, 126 are illustrated, it is understood that more or less can be used, as long as the combined surface area of the electrodes is less than the surface area of a surface adjacent the electrodes to prevent the electrosurgical device 110 from entering into a cut or puncture.

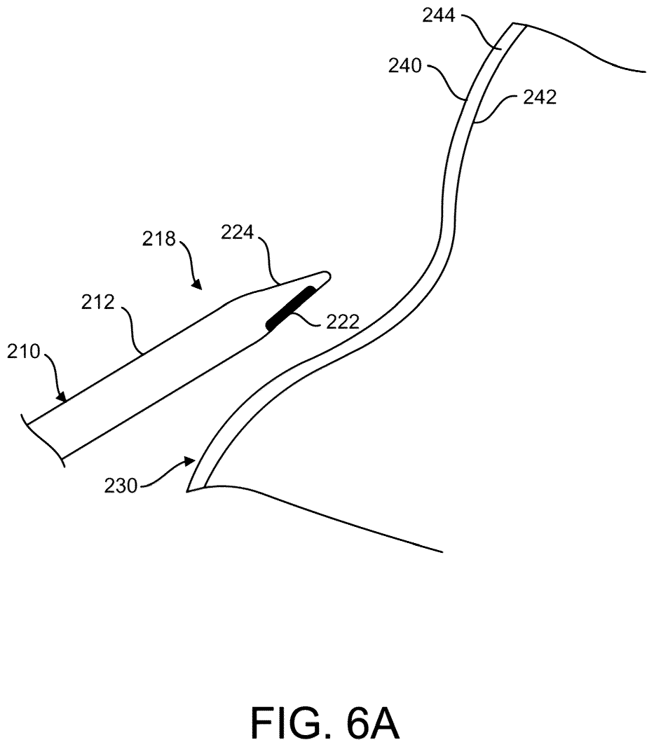

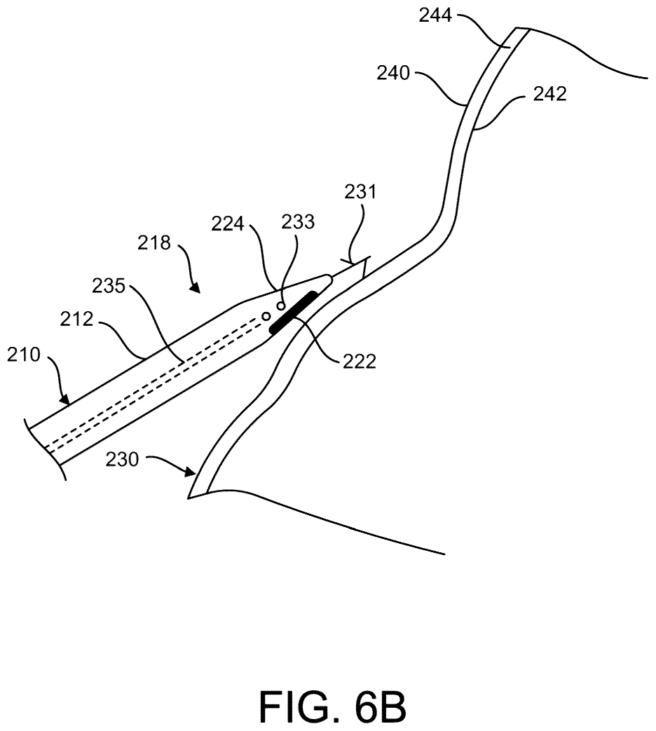

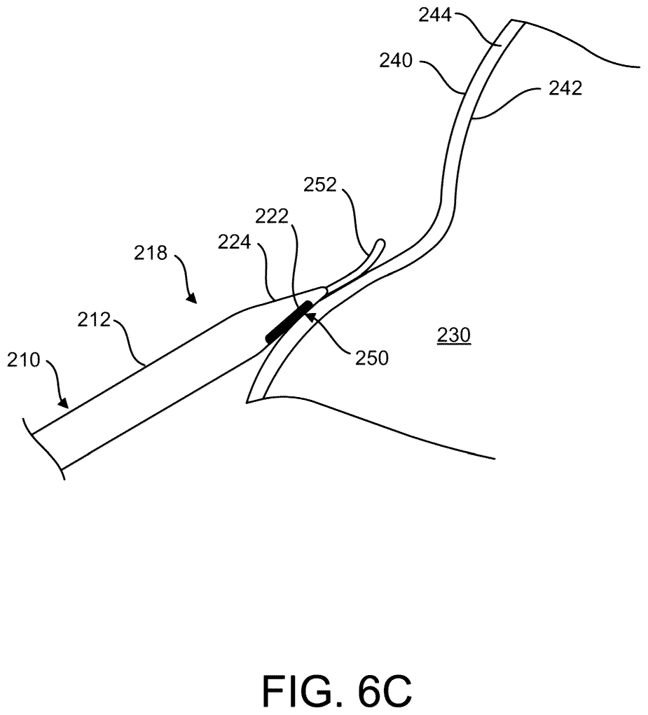

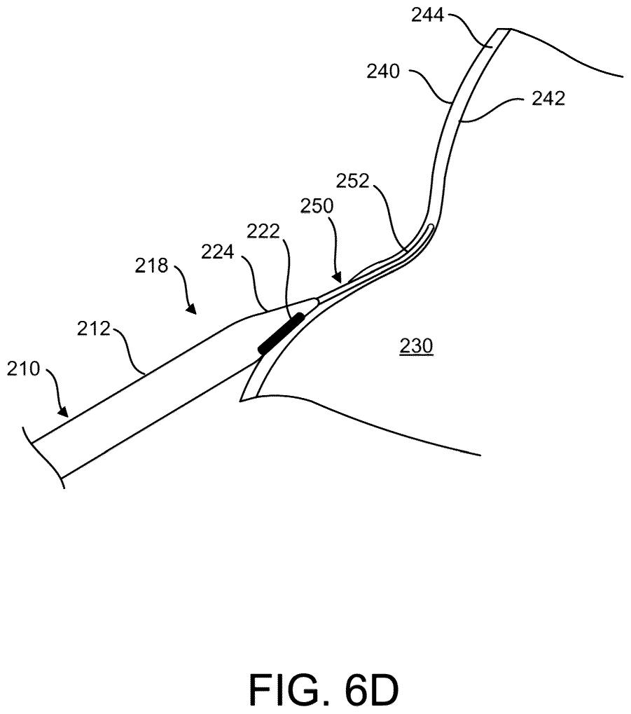

FIGS. 6A-6D illustrate a method of creating a pericardial puncture and accessing the pericardial space, in accordance with the disclosure. In FIG. 6A, an electrosurgical device 210 having at least one RF electrode 222 is introduced into the body and positioned adjacent the heart 230. The at least one RF electrode 222 is positioned on the distal portion 218 of an elongate shaft 212. The at least one RF electrode 222 is configured to have a surface area that is much smaller than a surface area of the distal portion 218 adjacent the at least one RF electrode. The at least one RF electrode 222 is a long electrode that extends along a tapered portion 224 of the distal portion 218.

In FIG. 6A, the electrosurgical device 210 is moved towards the heart 230. The heart 230 includes a pericardium 240 and a myocardium 242. The pericardium 240 and the myocardium 242 are separated by a pericardial space 244. In one aspect, pressing the electrosurgical device 210 into the heart 230 places the RF electrode 222 against the surface of the heart 230 and tents both the pericardium 240 and the myocardium 242. The pericardium 240 is the target tissue, that is the tissue that is desired to create a puncture or cut in, and the myocardium 242 is the non-target tissue.

In another aspect, as illustrated in FIG. 6B, the pericardium 240 can be brought towards the RF electrode 220 using a mechanical device such as a hook, snare, or barb 231 that can be advanced through a lumen of the electrosurgical device 210. The hook, snare, or barb 231 can capture the pericardium 240 and pull the pericardium 240 towards the RF electrode 220. In some aspects, the hook, snare, or barb 231 can be mounted on a surface of the electrosurgical device 210. The hook, snare, or barb 231 can include shape memory characteristics and move from a first configuration to a second configuration to pull tissue towards the RF electrode. In another aspect, the pericardium 240 can be brought towards the RF electrode 220 using suction. For example, the electrosurgical device 210 can include one or more apertures 233 adjacent the RF electrode 220. The one or more apertures 233 communicate to a source of suction through one or more lumens 235 extending towards the proximal end of the electrosurgical device 210.

In FIG. 6C, RF energy is applied to the at least one RF electrode 222. In one aspect, the energy is applied as a pulse for 1 second. In another aspect, the energy is applied as a 300 ms pulse using, for example, a 1s pulse mode (30% duty cycle). The application of energy to the at least one RF electrode 222 creates a slit, puncture, or cut 250 in the pericardium 240. A portion of the distal portion 218 prolapses through the slit, puncture, or cut 250 into the pericardial space 244. However, the surface area of the distal portion 218 surrounding the at least RF electrode 222 prevents “diving” of the electrosurgical device 210 in the direction of the force application on the tissue. As such, the at least one RF electrode 222 is prevented from slicing, cutting, or puncturing the myocardium 242, allowing introduction of the device into the pericardial space 244 without damaging the adjacent non-target tissue.

As illustrated in FIG. 6C, a mechanical guidewire 252 that extends from the distal end of the electrosurgical device 210 has a flexibility such that the guidewire 252 is unable to puncture the pericardium 240 and deflects off the pericardium 240. As such, if the electrosurgical device 210 is advanced at an angle towards the heart 230, the extended guidewire 252 deflects off the pericardium 240. In order to gain access to the pericardial space 244, the guidewire 252 must enter the slit, puncture, or cut 250 formed by the RF electrode 222. Upon entry into the slit, puncture, or cut 250, the guidewire 252 is axially aligned with the electrosurgical device 210. In one aspect, the guidewire 252 is formed of 038 stainless steel or 005 NiTi.

In FIG. 6D, the mechanical guidewire 252 is passed through the electrosurgical device 210 into the pericardial space 244. Introduction of the guidewire 252 into the pericardial space 244 allows for confirmation that access to the pericardial space 244 is achieved. The electrosurgical device 210 is then advanced along the guidewire 252 such that the tapered portion 224 can dilate the cut or puncture to allow for introduction of a larger medical device if so desired. In some aspects, a fluid such as a contrast medium may be introduced through the electrosurgical device 210 to the pericardial space 244.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An electrosurgical device for discretely cutting pericardium that is adjacent myocardium, the electrosurgical device comprising:
an elongate shaft having a proximal portion including a proximal end and a distal portion including a distal end; and
at least one electrode configured to cut the pericardium, the at least one electrode having a surface area and being located on the distal portion;
wherein the distal portion is electrically insulated from the at least one electrode and has a surface area significantly larger than the surface area of the at least one electrode, the distal portion being configured to prevent movement of the distal portion into a cut or puncture formed in the pericardium by application of energy to the at least one electrode.

2. The electrosurgical device of claim 1, wherein the elongate shaft includes a lumen extending from the proximal end to the distal end.

3. The electrosurgical device of claim 1, wherein the at least one electrode is formed of a wire having one or more rectangular, circular, oval, square, or polygonal cross-section.

4. The electrosurgical device of claim 1, wherein the at least one electrode is formed of an electrically conductive metal.

5. The electrosurgical device of claim 1, further comprising at least one lead configured to electrically connect the at least one electrode to a control system.

6. The electrosurgical device of claim 5, wherein the at least one lead extends proximally from the at least one electrode through a lead lumen to the proximal end of the elongate shaft.

7. The electrosurgical device of claim 5, wherein the at least one lead forms the at least one electrode.

8. The electrosurgical device of claim 1, further comprising a connector located at the proximal end configured for electrically connecting the electrosurgical device to a control system.

9. The electrosurgical device of claim 1, wherein the at least one electrode extends longitudinally along the outer surface of the distal portion.

10. The electrosurgical device of claim 9, wherein the outer surface tapers towards the distal end.

11. The electrosurgical device of claim 1, wherein the at least one electrode is located in a recess or groove along the distal portion.

12. The electrosurgical device of claim 1, wherein the at least one electrode has a surface area less than 1.0 mm².

13. The electrosurgical device of claim 1, wherein the at least one electrode is located on a distal face of the elongate shaft.

14. The electrosurgical device of claim 1, wherein the at least one electrode comprises a plurality of electrodes.

15. An electrosurgical device for discretely cutting pericardium that is adjacent myocardium, the electrosurgical device comprising:
an elongate shaft having a proximal portion including a proximal end and a tapered distal portion including a distal end; and
at least one electrode configured to cut the pericardium, the at least one electrode having a surface area and extending longitudinally along an outer surface of the distal portion;
wherein the distal portion is electrically insulated from the at least one electrode and has a surface area significantly larger than the surface area of the at least one electrode, the distal portion being configured to prevent movement of the distal portion into a cut or puncture formed in the pericardium by application of energy to the at least one electrode.

16. A method for discretely cutting target tissue using radiofrequency energy, the method comprising:
providing an electrosurgical device, the electrosurgical device comprising:
an elongate shaft including a proximal portion including a proximal end and a distal portion including a distal end; and
at least one electrode configured to cut the target tissue, the at least one electrode having a surface area and extending longitudinally along an outer surface of the distal portion;
wherein the distal portion is electrically insulated from the at least one electrode and has a surface area significantly larger than the surface area of the at least one electrode;
advancing the electrosurgical device to a target location within a patient; and
providing radiofrequency energy to the at least one electrode and forming a cut in the target tissue;
wherein the distal portion contacts tissue adjacent the cut in order to prevent movement of the distal portion into the cut.

17. The method of claim 16, wherein the distal portion tapers towards the distal end.

18. The method of claim 16, wherein the elongate shaft includes a lumen extending from the proximal end to the distal end.

19. The method of claim 16, wherein the target tissue is pericardium.

20. The method of claim 19, further comprising advancing a guidewire through the cut into a pericardial space.
